(19) United States
(12) Patent Application Publication
(10) Pub. No.: US 2023/0404970 A1
(43) Pub. Date: Dec. 21, 2023

(54) DIRECT DELIVERY OF VITAMINS TO REBALANCE GUT MICROBIOME AFTER EXPOSURE TO ANTIBIOTICS

(71) Applicant: DSM IP ASSETS B.V., Heerlen (NL)

(72) Inventors: Thanh-Van PHAM, Kaiseraugst (CH); Ateequr REHMAN, Kaiseraugst (CH); Robert STEINERT, Kaiseraugst (CH); Wilbert SYBESMA, Kaiseraugst (CH)

(21) Appl. No.: 18/250,811

(22) PCT Filed: Oct. 27, 2021
(86) PCT No.: PCT/EP2021/079869

§ 371 Date: Apr. 27, 2023

(30) Foreign Application Priority Data
Oct. 28, 2020 (EP) 20204235.4
Dec. 16, 2020 (EP) 20214437.4

(51) Int. Cl. A61K 31/375; A61K 31/4415; A61K 31/525; A61K 31/455; A61K 31/197; A61P 1/00; A61K 9/00

(52) U.S. Cl. CPC A61K 31/375; A61K 31/4415; A61K 31/525; A61K 31/455; A61K 31/197; A61P 1/00; A61K 9/0053

(57) ABSTRACT

This invention relates to the direct delivery to the intestine of a vitamin composition to restore a balanced population and metabolic profile of gut microbiome bacteria which has been disrupted due to exposure to antibiotics. The vitamin composition may comprise Vitamin C as a sole active agent; Vitamin C in combination with Vitamin B2 and/or B3; or Vitamin C in combination with Vitamin B2, Vitamin B3, Vitamin B5, Vitamin B6 and Vitamin B9.

### BRIEF DESCRIPTION OF THE INVENTION

This invention relates to the direct delivery to the intestine of a vitamin composition to restore a balanced population and metabolic profile of gut microbiome bacteria which has been disrupted due to exposure to antibiotics. The vitamin composition may comprise Vitamin C as a sole active agent; Vitamin C in combination with Vitamin B2 and/or B3; or Vitamin C in combination with Vitamin B2, Vitamin B3, Vitamin B5, Vitamin B6 and Vitamin B9.

### BACKGROUND OF THE INVENTION

Direct delivery of various vitamins and other active ingredients to the gut has been described. See, e.g., U.S. Pat. No. 9,433,583 B2 directed to a colon-targeted single dosage form comprising vitamin D and optionally further vitamins for preventing colorectal adenomatous polyps and colorectal cancer and WO2014/070014 directed to the use of riboflavin (Vitamin B2) to stimulate the population of Faecalibacterium prausnitzii.

The use of broad spectrum antibiotics, such as penicillins/cephalosporine, fluoroquinolones, and clindamycin will alter the populations of the gut microbiome and metabolic profile, a condition known as microbial dysbiosis.

Microbial dysbiosis leads to altered metabolism of key intestinal nutrients, whose build-up induces an osmotic diarrhea. Moreover, microbial dysbiosis reduces colonization resistance and favors subsequent infection with pathogenic bacteria (e.g. Clostridium difficile, Clostridium perfringens, Klebsiella oxytoca, Staphylococcus aureus, Salmonella spp.).

Common side effects associated with the use of antibiotics may range from minor annoyances to extremely serious. They can include: flatulence, bloating, antibiotic associated diarrhea (AAD), occurring in to 35% of patients who receive antibiotics, malabsorption of nutrients, acid reflux, irritable bowel syndrome, acne, autoimmune diseases, yeast infections, and mental illnesses, including anxiety and depression. AAD itself may be severe enough to be life threatening.

Current therapeutic measures include stabilization of the gut microbiome against the threat of dysbiosis from antibiotics. One approach is to advise patients to take probiotics to help rebalance the microbiota. However, the rebalancing can take time, and the desirable bacterial do not always colonize harmoniously, or the pathogenic bacteria may populate the gut at a faster rate. Dosages of probiotics have to be timed carefully—at least two hours before and after an antibiotic dose in order to be effective, and this regime is not always convenient to follow. Further some people are sensitive to probiotics and are advised to avoid histamine-producing strains, such as Lactobacillus reuteri, L. casei, and L. bulgaris.

Another recommendation is to include dietary fibres or prebiotics such as Fructooligosaccharides (FOS) into one's diet. Moreover, simple sugar intakes can be reduced, and instead increase the intake of starches which are resistant to digestion. These include unroasted cashew, raw potato starch, raw green bababas, and raw plantain. These changes can decrease the occurrence of yeast (especially Candida albans) infections. However, this is also difficult for many people to follow, and if one is already suffering from irritable bowel syndrome or Crohn's Disease, the resistant starch can actually aggravate the condition.

It would be desirable to have an all natural, easy to use composition which help to rebalance the microbiome after exposure to antibiotics.

### DETAILED DESCRIPTION OF THE INVENTION

It has been found, in accordance with this invention that a composition comprising Vitamin C as the sole active ingredient when administered directly to the gut of a person exposed to antibiotics, can prevent and/or treat microbial dysbiosis, increase short chain fatty acids, decrease redox potential, and favor the repopulation of the gut with a diverse bacterial flora, i.e. increase microbial diversity. The Vitamin C may also be combined with other active ingredients, such as Vitamin B2 and Vitamin B3. Also, a combination of Vitamin C, Vitamin B2, Vitamin B3, Vitamin B5, Vitamin B6 and Vitamin B9 (herein after referred to as “Vitamin Mix”) is also effective.

While not wishing to be bound by theory, it is believed that the Vitamin C may work by multiple means. First, it (and the other ingredients of Vitamin Mix) may support bacterial metabolism, and as a result, enhance the production of Short Chain Fatty Acids (SCFAs), thus, contributing to the overall anti-pathogenic effect observed. In addition, particularly Vitamin C and vitamin B2, which have direct and indirect antioxidant properties, may decrease the local availability of oxygen to pathogens, thus limiting their growth. Also, there will be a lowering of the gut redox potential that will limit the growth of undesirable aerotolerant microbes and stimulate the growth of beneficial strict anaerobic species. Further, particularly Vitamin C (but also indirectly via an increased production of SCFA) may lower the physiological pH in the intestine, which may contribute to the growth inhibitory effect.

### Microbial Dysbiosis

Thus, one embodiment of this invention is a method of preventing or treating microbial dysbiosis associated with antibiotic treatment comprising administering a composition comprising Vitamin C directly to the large intestine of a person who is exposed to an antibiotic. Another embodiment of this invention is a method of preventing or treating microbial dysbiosis associated with antibiotic treatment comprising administering a composition comprising Vitamin C, Vitamin B2 and Vitamin B3 directly to the large intestine of a person who is exposed to an antibiotic. Another embodiment of this invention is a method of preventing or treating microbial dysbiosis associated with antibiotic treatment administering a composition comprising Vitamin C, Vitamin B2, Vitamin B3, Vitamin B5, Vitamin B6 and Vitamin B9 (“Vitamin Mix”) directly to the large intestine of a person who is exposed to an antibiotic.

One embodiment of this invention is the use of a directly delivered composition comprising Vitamin C to prevent or treat antibiotic associated microbial dysbiosis in a person who is exposed to an antibiotic. In some embodiments the composition used comprises Vitamin C, Vitamin B2 and Vitamin B3. In some embodiments, a Vitamin Mix is used to prevent or treat antibiotic associated microbial dysbiosis in the distal gut. Another embodiment is the use of directly delivered Vitamin Mix to prevent or treat antibiotic associated microbial dysbiosis in the distal gut. Another embodiment is the use of directly delivered Vitamin Mix to prevent or treat antibiotic associated microbial dysbiosis.

As symptoms associated with antibiotic associated microbial dysbiosis include: flatulence, bloating, antibiotic associated diarrhea (AAD), malabsorption of nutrients, acid reflux, irritable bowel syndrome, acne, autoimmune diseases, yeast infections, anxiety and depression, another embodiment of this invention is a method of preventing at least one of the foregoing adverse consequences by directly delivery to the large intestine of a person who is exposed to antibiotics, at least one active ingredient selected from the group consisting of: Vitamin C, Vitamin B2, Vitamin B3, Vitamin B5, Vitamin B6 and Vitamin B9, and mixtures thereof.

Thus, another embodiment is a method of preventing, reducing the risk or delaying the onset of a disease or adverse condition; methods of treating a disease or adverse condition; and methods of meeting the nutritional needs of a person experiencing a disease or adverse condition, wherein the disease or adverse condition is associated with antibiotic associated microbial dysbiosis. In some embodiments, the disease or adverse condition is selected from the group consisting of: flatulence, bloating, antibiotic associated diarrhea (AAD), malabsorption of nutrients, acid reflux, irritable bowel syndrome, acne, autoimmune diseases, yeast infections, anxiety and depression. In some embodiments the active ingredient is Vitamin C. In some embodiments it is Vitamin C, Vitamin B2 and Vitamin B3; and in some embodiments it is the Vitamin Mix.

Another embodiment is the use of directly delivered Vitamin C; Vitamin C, Vitamin B2 and Vitamin B3; or Vitamin Mix to prevent or treat microbial dysbiosis associated with exposure to antibiotics. Another embodiment is the use of a directly delivered active ingredient selected from the group consisting of Vitamin C; Vitamin C, Vitamin B2 and Vitamin B3; or Vitamin Mix to prevent or treat adverse conditions of microbial dysbiosis associated with exposure to antibiotics. In some embodiments the adverse conditions are selected from the group consisting of: flatulence, bloating, antibiotic associated diarrhea (AAD), malabsorption of nutrients, acid reflux, irritable bowel syndrome, acne, autoimmune diseases, yeast infections, anxiety and depression. In some embodiments the treatment or prevention is non-therapeutic.

Another embodiment, a directly delivered active ingredient selected from the group consisting of Vitamin C; Vitamin C, Vitamin B2 and Vitamin B3; or Vitamin Mix is used to make a medicament for the prevention or treatment of microbial dysbiosis. In some embodiments the medicament is used to prevent or treat an adverse condition selected from the group consisting of: flatulence, bloating, antibiotic associated diarrhea (AAD), malabsorption of nutrients, acid reflux, irritable bowel syndrome, acne, autoimmune diseases, yeast infections, anxiety and depression.

### BRIEF DESCRIPTION OF THE FIGURES

FIG. 1: shows the experimental setup of the in vitro fermentation experiment.

FIG. 2: Effect of the combination of vitamins (“Vitamin Mix”), FOS and Vitamin Mix plus FOS compared to a blank control on SCFA production in the presence of antibiotic treatment: (A) Data are expressed as average and STDEV of propionate production (mM) during the stabilization period (STAB), the antibiotic period (AB), and the treatment period (3 w) (n=2). * indicates statistically significant differences relative to STAB, p<0.05; # indicates statistically significant differences relative to control, p<0.05. (B) Data are expressed as average and STDEV of butyrate production (mM) during the stabilization period (STAB), the antibiotic period (AB), the treatment period (3 w) (n=2). * indicates statistically significant differences relative to STAB, p<0.05; # indicates statistically significant differences relative to control, p<0.05; § indicates statistically significant differences relative to FOS and FOS+Vitamin Mix, p<0.05.

FIG. 3: Effect of the combination of vitamins (“Vitamin Mix”), FOS and Vitamin Mix plus FOS compared to a blank control on redox potential (mV) in the colon (n=1). STAB: stabilization period, AB: antibiotic period, 3 w: treatment period.

FIG. 4: Effect of the combination of vitamins (“Vitamin Mix”), FOS and Vitamin Mix plus FOS compared to a blank control on reciprocal Simpson diversity index in the colon. Data are expressed as average and STDEV of diversity index during the stabilization period (STAB), the antibiotic period (AB), the treatment period (3 w) (n=2). $ indicates differences relative to control, p<0.1

### DEFINITIONS

As used throughout, the following definitions apply:

The term “Vitamin Mix” means the combination of Vitamin C, Vitamin B2, Vitamin B3, Vitamin B5, Vitamin B6 and Vitamin B9.

The term “vitamin B2” which can be used interchangeably with “riboflavin”, includes riboflavin and esters thereof, in particular riboflavin-5′-phosphate and other pharmaceutically acceptable forms.

The term “vitamin C” which can be used interchangeably with “ascorbic acid” also includes pharmaceutically acceptable salts thereof (e.g. sodium ascorbate and calcium ascorbate) and pharmaceutically acceptable esters thereof (in particular ascorbyl palmitate) and other pharmaceutically acceptable forms.

The term “Vitamin B3” which can be used interchangeably with “niacinamide” and “niacin” also includes nicotinic acid and other pharmaceutically acceptable forms.

The term “Vitamin B5” which can be used interchangeably with “pantothenic acid” also includes coenzyme A, phosphopantetheine, pantetheine and other pharmaceutically acceptable forms.

The term “Vitamin B6” which can be used interchangeably with “pyridoxine” also includes pyridoxine phosphate, pyridoxal, pyridoxal phosphate and other pharmaceutically acceptable forms.

The term “Vitamin B9” which can be used interchangeably with “Folic Acid” also includes folate, 5-methyl-tetrahydrofolate, monoglutamate folate, polyglutamate folate and other pharmaceutically acceptable forms.

“Direct delivery” or “directly delivered” means that the vitamin or combination of vitamins is administered in a manner such that the vitamin(s) is not absorbed in the stomach and/or small intestine; rather the vitamin(s) is present in the distal intestinal tract, preferably the large intestine, where it is available to the microbiome. The vitamin(s) are not part of a person's usual daily nutritional requirements (generally obtained through diet and conventional vitamin supplementation), and are administered in excess thereof. For human use, the preferred method is through a form which delays release until the large intestinal tract is reached. Alternatively, is included a method of administering a large enough dose so that only a portion of the vitamin delivered is absorbed in the proximal small intestine, and the remainder which is an effective dose, is available to the large intestinal tract; although not preferred, this method of delivery can be used for humans as well.

“Prevent” can include lessening the risk of an adverse condition occurring, lessening the symptoms of an adverse condition, lessening the severity of an adverse condition and prolonging the time for occurrence of an adverse condition.

“Exposed to antibiotics” means that a person has either received an antibiotic within two weeks prior to receiving the directly delivered vitamin(s) of this invention; or will be receiving an antibiotic within two weeks of receiving the directly delivered vitamins of this invention.

Another embodiment of this invention is a medical food for persons who is exposed to an antibiotic which meets the nutritional needs of the person and comprises an active ingredient selected from the group consisting of Vitamin C, the combination of Vitamin C, Vitamin B2 and B3, and Vitamin Mix; and the active ingredient is formulated for direct delivery.

Thus, another aspect of this invention is a vitamin combination formulation comprising an active ingredient selected from the group consisting of Vitamin C, the combination of Vitamin C, Vitamin B2 and B3, and Vitamin Mix, and excipients; and said formulation is characterized in that the active ingredient is delivered directly to the gut microbiome in the large intestine, and is used for addressing the nutritional needs of a patient experiencing microbial dysbiosis.

The antibiotic may be any antibiotic, including those which are known as broad spectrum antibiotics. Examples include, but are not limited to: penicillins/cephalosporine, fluoroquinolones, and clindamycin.

As antibiotics are known to affect the makeup of the gut microbiome for a prolonged period of time, the vitamin(s) composition may be administered up to two weeks prior to the administration of an antibiotic, concurrently with the administration of an antibiotic; or within one week, two weeks, within 4 weeks, or up to 90 days after administration of the antibiotic. A person “exposed to antibiotics” is someone who is currently receiving antibiotic treatment, who will be receiving antibiotic treatment within 2 weeks of receiving direct delivery of the vitamin(s) of this invention, or has already received antibiotic treatment within one week, two weeks, within 4 weeks, or up to 90 days after administration of the vitamin(s) of this invention.

### Additional Active Ingredients

The vitamin(s) of this invention may be used as the sole active ingredients, or may be combined with other active ingredients, such as conventional drug therapies, prebiotics, probiotics, and the like.

### Doses:

The dosages used herein are intended to be in addition to the active ingredients that is ingested for general nutrition purposes. Instead, they act upon the gut microbiome environment as a whole, at the genus, species and strain level of the gut microbes. The active agents are not intended primarily to be metabolized directly by the animal, including the human rather they are intended primarily to be utilized by the bacterial population of the colon. Therefore, the amounts reported below would be consumed by the animal in addition to the usual diet, but as they are not directly available to the animal due to their delayed release.

Preferably, Vitamin B2 can be administered in an amount such that its local concentration in the colon is at least 0.01 g/L, preferably at least 0.1 g/L more preferably at 0.125 g/L. Preferred local concentrations in the colon range from about 0.1 g/L to about 0.5 g/L or from about 0.1 g/L to about 0.2 g/L, preferably about 0.125 g/L. Specific dosages per day can range up to 200 mg/day, preferably 5-100 mg/day, more preferably from 10-50 mg/day.

Preferably, Vitamin C can be administered in an amount such that its local concentration in the colon is at least 0.05 g/L, preferably at least 0.1 g/L, most preferably at least 2 g/L. Preferred local concentrations in the colon range from about 0.05 g/L to about 1.5 g/L, more preferably from about 0.5 g/L to about 1 g/L, most preferably from about 0.8 g/L to about 0.9 g/L. Specific dosages per day can range up to 2000 mg/day, preferably 100-2000 mg/day; more preferably 200-1000 mg/day.

Preferably, vitamin B5 can be administered in an amount such that its local concentration in the colon is at least 0.01 g/L, preferably at least 0.02 g/L, most preferably at least 0.04 g/L. Preferred local concentrations in the colon range from about 0.005 g/L to about 0.04 g/L, more preferably from about 0.015 g/L to about 0.025 g/L. Specific dosages per day can range up to up to 50 mg/day, preferably 1-50 mg/day; more preferably 5-25 mg/day.

Preferably, vitamin B3 can be administered in an amount such that its local concentration in the colon is at least 0.01 g/L, preferably at least 0.02 g/L, most preferably at least 0.04 g/L. Preferred local concentrations in the colon range from about 0.005 g/L to about 0.04 g/L, more preferably from about 0.015 g/L to about 0.025 g/L. Specific dosages per day can range up to up to 50 mg/day, preferably 1-50 mg/day; more preferably 5-25 mg/day.

Preferably, vitamin B6 can be administered in an amount such that its local concentration in the colon is at least 0.003 g/l, preferably at least 0.007 g/l, most preferably at least 0.01 g/L. Preferred local concentrations in the colon range from about 0.003 g/l to about 0.05 g/l, more preferably from about 0.008 g/l to about 0.03 g/l, most preferably from about 0.01 g/l to about 0.02 g/l. Specific dosages per day can range up to 20 mg/day, preferably 0.5-20 mg/day, more preferably 1.7-9 mg/day.

Preferably, vitamin B9 can be administered in an amount such that its local concentration in the colon is at least 0.001 g/l, preferably at least 0.002 g/l, most preferably at least 0.003 g/l. Preferred local concentrations in the colon range from about 0.001 g/l to about 0.01 g/l, more preferably from about 0.003 g/l to about 0.008 g/l, most preferably from about 0.005 g/l to about 0.007 g/l. Specific dosages per day can range up to 5 mg/day, preferably 0.1-5 mg/day, more preferably 0.4-2 mg/day.

### Formulations

A suitable formulation may include a high enough dosage so that a portion of the combination of vitamins is absorbed normally, but the remainder is available to the gut microbiome in the intestine at an effective amount. Other formulations include non-oral routes, such as via suppositories or injections. Preferred formulations are delayed release oral formulations.

A used herein, “delayed release” refers to the release of the active agent at a time later than immediately after administration. Preferably, “delayed release” means delivery of the active agent, upon oral administration, to the large intestine, preferably the colon, in a delayed manner relative to an immediate release formulation.

An “enteric layer” is a layer surrounding a core, wherein the core comprises the active agent and the layer confers resistance to gastric juice. An “enteric shell” is a shell or matrix surrounding or encapsulating the active agent, wherein the shell confers resistance to gastric juice. Alternatively, a matrix-based delivery system can be used. Matrix based systems have no discrete layer of coating material but the active agent is more or less homogeneously distributed within the matrix. Further, there are colon-release systems that embed the active agent in e.g. in a fiber matrix (enzyme-triggered) and an enteric coating on top.

In a preferred embodiment for humans, the formulation of the present invention is a solid dosage form for oral administration. The formulation may be in the form of a capsule, pellet, bead, sphere, mini spheres, tablet, mini tablet, or granule, optionally coated with a delayed release coating or shell that prevents the release of the active agent before the small intestine, preferably before the colon.

Coating, shell, or matrix materials for the delayed release of the active agent, in particular for targeted release in the ileum or the large intestine, upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating or shell materials from different categories are commonly used in combinations. Coating or shell materials of these three different categories for targeting to the large intestine have been reviewed for example in Bansal et al. (Polim. Med. 2014, 44, 2,109-118). In one embodiment of the present invention the delayed release coating comprises at least one component selected from coating materials that disintegrate pH-dependently, coating materials that disintegrate time-dependently, coating materials that disintegrate due to enzymatic triggers in the intestinal environment (e.g. in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, and Eudragit® FS30D).

Coating materials that disintegrate time-dependently include Eudragit® RL, Eudragit® RS, and ethylcellulose.

Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

In one embodiment the formulation comprises an enteric capsule, filled with a composition comprising the active agent. The enteric capsule confers resistance against the acidic environment of the stomach. For example, softgel formulations may deliver the active agent in solution and yet offer advantages of solid dosage forms. Softgel capsules are particularly suited for hydrophobic active agents which do not dissolve readily in water. Vitamin K and omega-3 fatty acids are preferably formulated in softgel capsules.

In another embodiment, the formulation is a tablet comprising (i) a core comprising the active agent, and (ii) a delayed release coating such as an enteric coating. This may be a hard gel capsule.

The release of the active agent(s) may be delayed until the small intestine. In another embodiment, the release of the active agent(s) is delayed until the distal small intestine. In yet another embodiment, the release of the active agent(s) is delayed until the large intestine including the colon.

The following non-limiting Examples are presented to better illustrate the invention

### EXAMPLES

### Example 1

### Experimental Setup

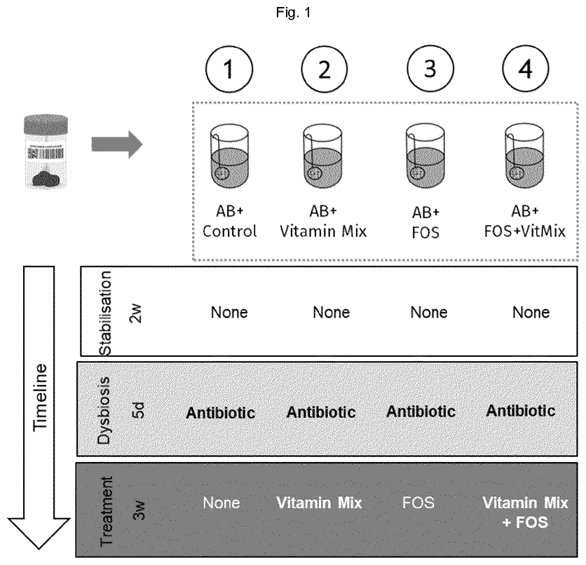

### In Vitro Fermentation Study (FIG. 1)

The properties of a vitamin combination (Vitamin C, Vitamin B2, Vitamin B3, Vitamin B5, Vitamin B6 and Vitamin B9) (referred to as “Vitamin Mix”), Fructooligosaccharide (“FOS”) a recognized prebiotic, a combination of Vitamin Mix and FOS, and a blank control were evaluated in the presence or absence of antibiotic intervention in a fermentation configuration using the microbiota of a healthy adult human donor.

In practice, upon inoculation with a fecal microbiota of a human adult, these reactors simulate the transverse colon (pH 6.15-6.4; retention time=32 h; volume of 800 mL).

The fermentation experiment for this study consisted of three stages:
Stabilization period: After inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the faecal inoculum. Analysis of samples at the end of this period allows to determine the baseline microbial community composition and activity in the different reactors.
Antibiotic period: In this four-day period, antibiotic treatment was initiated in arms (1) to (4) of the SHIME and clindamycin was dosed to the colon reactors at a final concentration of 33.9 ppm for a period of 3 days. On day 4, analysis of samples allows to determine the baseline microbial community composition and activity in the different reactors after antibiotic treatment.
Treatment period: During this three-week period, the SHIME reactor was operated under nominal conditions, but with a diet supplemented with the test product. Samples taken from the colon reactors in this period allow to investigate the specific effect on the resident microbial community composition and activity. For the blank control condition, the standard SHIME nutrient matrix was further dosed to the model. Analysis of samples of these reactors allow for determination of the normal microbial community composition and activity in the different reactors, which will be used as a reference for evaluating the treatment effects.
   Treatment with Vitamin Mix includes two weeks of treatment at low dose and one week of treatment at high dose (Table 1).

TABLE 1
Dosage of the different compounds of the test product in the in vitro experiment.

| | Products | Low dose | | | | High dose | | | |
|---|---|---|---|---|---|---|---|---|---|
| Vitamin | Riboflavin (Vitamin B2) | 10 | mg/d | 0.02 | g/L | 50 | mg/d | 0.08 | g/L |
| Mix | Ascorbic Acid (Vitamin C) | 200 | mg/d | 0.33 | g/L | 1000 | mg/d | 1.67 | g/L |
| | Niacinamide (Vitamin B3) | 5 | mg/d | 0.01 | g/L | 25 | mg/d | 0.04 | g/L |
| | Pantothenic acid (Vitamin B5) | 5 | mg/d | 0.01 | g/L | 25 | mg/d | 0.04 | g/L |
| | Pyridoxine (Vitamin B6) | 1.7 | mg/d | 0.003 | g/L | 9 | mg/d | 0.01 | g/L |
| | Folic acid (Vitamin B9) | 0.4 | mg/d | 0.001 | g/L | 2 | mg/d | 0.003 | g/L |

### Results

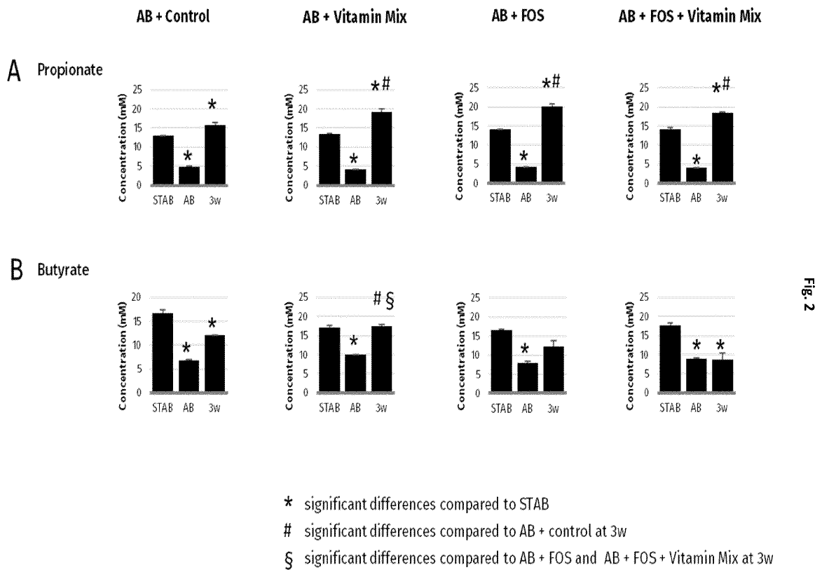

### Effect on SCFA Production (FIG. 2):

Antibiotic treatment significantly decreased propionate and butyrate levels in all treatment arms when compared with the stabilization period.
Treatment with Vitamin Mix resulted in significantly higher propionate levels as compared to the control after 3 weeks (p<0.05). Similar effects were observed with FOS and with FOS+Vitamin Mix.
Treatment with Vitamin Mix also resulted in a complete recovery of butyrate levels with significantly higher butyrate levels as compared to control (p<0.05), FOS (p<0.05) and FOS+Vitamin Mix

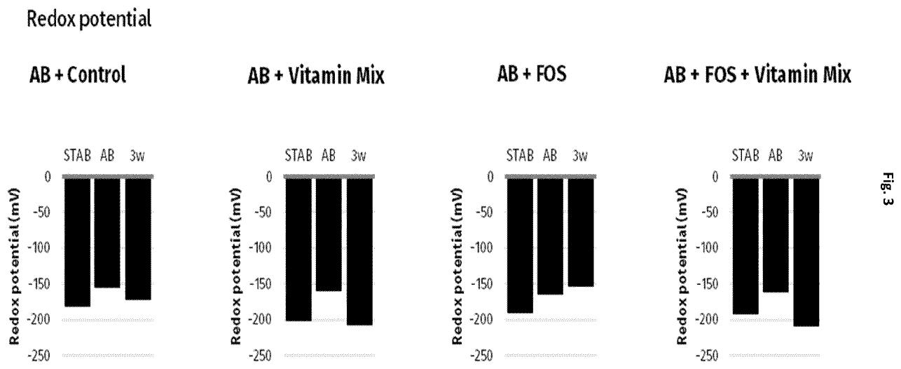

### Effect on Redox Potential (FIG. 3):

Antibiotic treatment increased the redox potential in all treatment arms when compared with the stabilization period.
Treatment with Vitamin Mix resulted in a full recovery of a low redox potential which was in contrast to what was observed with control and FOS. With the control there was no full recovery of a low redox potential, with FOS, the redox potential even further increased at 3 weeks.
Treatment with a combination of FOS and Vitamin Mix also resulted in a full recovery of a low redox potential similar to Vitamin Mix alone

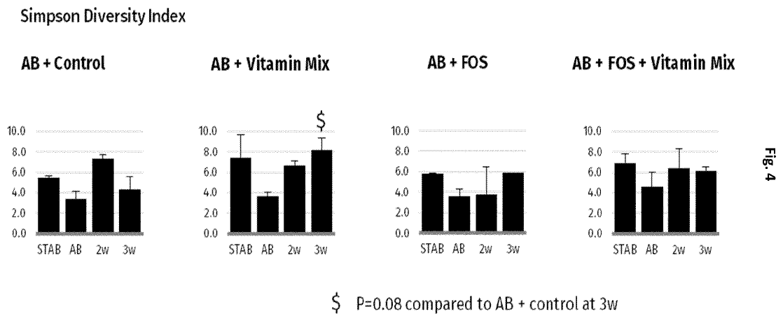

### Effect on Microbial Diversity (FIG. 4):

Antibiotic treatment decreased microbial diversity in all treatment arms when compared with the stabilization period.
Treatment with Vitamin Mix resulted in a full recovery in microbial diversity which was not the case with control. Similar effects were also observed with FOS and FOS+Vitamin Mix alone.
Treatment with Vitamin Mix resulted in higher microbial diversity as compared to the control after 3 weeks (p=0.08).
Results on SCFA production, redox potential and bacterial diversity derived from different donors.

1. A method of preventing or treating a symptom of microbial dysbiosis associated with antibiotic treatment comprising administering a composition comprising Vitamin C directly to the large intestine of a person who is exposed to an antibiotic.

2. The method of claim 1, wherein the composition further comprises Vitamin B2 and Vitamin B3.

3. The method of claim 1 wherein the composition comprises composition comprising Vitamin C, Vitamin B2, Vitamin B3, Vitamin B5, Vitamin B6 and Vitamin B9 (“Vitamin Mix”).

4. The method of claim 1 wherein the symptom is selected from the group consisting of flatulence, bloating, antibiotic associated diarrhea (AAD), malabsorption of nutrients, acid reflux, irritable bowel syndrome, acne, autoimmune diseases, yeast infections, anxiety and depression.

5. The method of claim 1 wherein the method is characterized by at least one of the following:
an enhanced the production of Short Chain Fatty Acids (SCFAs);
a decrease the local availability of oxygen to pathogens;
a lowering of the gut redox potential;
a lowering the physiological pH in the intestine; or
an increase in microbial diversity.

6. Use of a composition comprising Vitamin C to prevent or treat a symptom of microbial dysbiosis, wherein the composition is directly delivered to the large intestine of a person who is exposed to an antibiotic.

7. Use according to claim 6 wherein the composition is further characterized by comprising Vitamin B2, and Vitamin B3.

8. Use according to claim 6, wherein the composition comprises Vitamin C, Vitamin B2, Vitamin B3, Vitamin B5, Vitamin B6 and Vitamin B9 (“Vitamin Mix”).

9. Use according to claim 6 wherein the symptom is selected from the group consisting of: flatulence, bloating, antibiotic associated diarrhea (AAD), malabsorption of nutrients, acid reflux, irritable bowel syndrome, acne, autoimmune diseases, yeast infections, anxiety and depression.

10. Use according to claim 6 wherein characterized by at least one of the following:
an enhanced the production of Short Chain Fatty Acids (SCFAs);
a decrease the local availability of oxygen to pathogens;
a lowering of the gut redox potential;
a lowering the physiological pH in the intestine; or
an increase in microbial diversity.
